(19) United States
(12) Patent Application Publication
(10) Pub. No.: US 2023/0348353 A1
(43) Pub. Date: Nov. 2, 2023

(54) METHOD FOR RECOVERING MALONATE FROM A FERMENTATION BROTH

(71) Applicant: CARGILL, INCORPORATED, Wayzata, MN (US)

(72) Inventors: Timothy Walter ABRAHAM, Minnetonka, MN (US); Ravi R. GOKARN, Omaha, NE (US); Nolan R. MENTE, Pella, IA (US)

(73) Assignee: CARGILL, INCORPORATED, Wayzata, MN (US)

(21) Appl. No.: 17/999,744

(22) PCT Filed: May 28, 2021
(86) PCT No.: PCT/US2021/034881

§ 371 Date: Nov. 23, 2022

Related U.S. Application Data
(60) Provisional application No. 63/032,034, filed on May 29, 2020.

(51) Int. Cl. C07C 51/48

(52) U.S. Cl. CPC C07C 51/48

(57) ABSTRACT

The disclosure relates to a method for recovering a malonate comprising a compound of the formula HOC(O)CH₂C(O)OM, wherein M is a Group I alkali metal cation or ammonium cation, the method comprising the steps of a) removing water from a fermentation broth comprising malonate to obtain an aqueous concentrate; and b) recovering the malonate comprising the compound of the formula I from the aqueous concentrate.

### CROSS-REFERENCE TO RELATED APPLICATIONS

This application claims the benefit of U.S. Provisional Patent Application No. 63/032,034, filed May 29, 2020, which is incorporated by reference herein in its entirety.

### FIELD OF THE DISCLOSURE

The disclosure relates to a process for recovering malonate from a fermentation broth. More particularly, the disclosure relates to a process for recovering mono-salts of malonate (and optionally di-salts of malonate and malonic acid) from a fermentation broth.

### BACKGROUND

Fermentation processes are used commercially at large scale to produce organic molecules such as ethanol, citric acid and lactic acid. In those processes, a carbohydrate is fed to an organism that metabolizes the carbohydrate to a desired fermentation product. The carbohydrate and organism are selected together so that the organism can efficiently use the carbohydrate to form the desired product in good yield. It is becoming more common to use genetically engineered organisms in these processes, in order to optimize yields and process variables, or to enable a particular fermentation product to be produced.

Fermentation processes have also been applied to produce malonic acid. But the compound's high water solubility and decreased stability at low pH and high temperatures makes the isolation of malonic acid from a fermentation broth more challenging than other organic acids, such as lactic acid and citric acid, since malonic acid tends to decarboxylate under those conditions.

Two methods are generally used to isolate an organic acid, such as malonic acid, when it is produced by fermentation. One method involves the conversion of the organic acid to a completely protonated form, often referred to as the free acid form, followed by isolation of the free acid by processes such as distillation, crystallization, chromatography or extraction with an extractant and/or solvent. Another method involves conversion of the organic acid into a completely neutralized form and recovering the ‘fully-neutralized’ carboxylate salt by crystallization/precipitation. But these methods often suffer from, among other things, low recovery yields when applied to malonic acid.

### SUMMARY

The methods described herein relate to the recovery of malonate from a fermentation broth produced by a microorganism that is able to produce malonate from a fermentable carbon source. The methods described herein can be performed at a commercially viable level in high recovery yields; and while minimizing the decarboxylation of the malonate.

The disclosure relates to methods for recovery of malonate, either by direct crystallization from a malonate containing aqueous solution, or by solvent-mediated precipitation from the aqueous solution, typically at a pH between 2.5 and 5.0 (for example from pH of from 2.7 to 4.5, 3 to 4, from 3.0 to 3.5). The malonate recovered typically comprises at least forty percent by weight (40 wt %) of the compound of formula I:

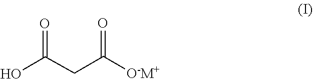

wherein M is a Group I alkali metal or ammonium, less than fifty percent by weight (50 wt %) malonic acid (for example, less than forty percent by weight (40 wt %) malonic acid), and twenty five percent by weight (25 wt %) or less of the compound of formula II:

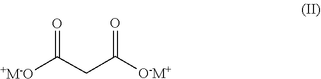

wherein M is a Group I alkali metal, ammonium, or mixtures, thereof.

The methods described herein provide a method for recovering malonate in high yield with a minimum number of precipitations. When used herein, precipitation includes crystallization and precipitation by other means known to one of ordinary skill in the art. For example, typically malonate can be recovered at a yield of typically at least 75 percent with less than three precipitations, and preferably at a yield of at least 80 percent with two precipitations (above 85 or above 90 percent when the concentration of malonate in aqueous concentrate is at least 500 g/kg), and in some instances a yield of at least 65 percent, at least 70 percent with one precipitation. The precipitate/crystals obtained from each round of recovery is sometimes referred to as a “crop”. For example, the precipitate of malonate recovered from the first recovery is sometimes referred to as the “first crop” and the second precipitate of malonate recovered from the second recovery is sometimes referred to as the “second crop.” Additional precipitate recovered from additional precipitations would be referred to similarly (e.g third crop, fourth crop, etc. Once recovered, the malonate can readily be converted into malonic acid, diesters of malonic acid and other derivatives of malonic acid/diesters of malonic acid, by methods known by one of skill in the art.

### BRIEF DESCRIPTION OF THE FIGURES

The drawings illustrate generally, by way of example, but not by way of limitation, various embodiments discussed in the present document.

FIGS. 1A-1D are flow diagrams of examples of the methods described herein.

### DESCRIPTION

The disclosure relates generally to a method for recovering malonate. As used herein, the term “malonate” includes malonic acid (formula (III), below) and salts thereof. When referring to malonate concentration (e.g. malonate recovered) herein we are referring to the concentration of malonic acid equivalents (i.e. the sum of: monosalts of malonic acid (formula I), disalts of malonic acid (formula II), and free malonic acid (formula III) excluding metals ions and other cations.

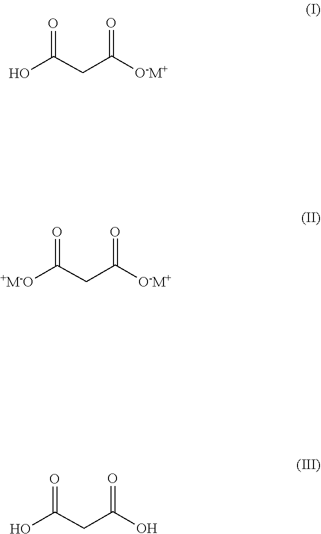

Wherein M is a Group I alkali metal (such as Li, Na, K, ammonium (such as NH4), or mixtures, thereof.

The method comprising the steps of:
a) removing water from a fermentation broth comprising malonate (typically, the fermentation broth has a concentration of at least 30 g/L, at least 40 g/L, at least 50 g/L and less than 300 g/L malonate (for example, from 60 g/L to 250 g/L malonate, preferably from 70 g/L to 200 g/L malonate), to obtain an first aqueous concentrate having a malonate concentration typically of at least 400 g/kg (for example, at least 500 g/kg), and typically between 400 g/kg and 775 g/kg, for example 450 g/kg to 700 g/kg (preferably between 500 g/kg and 600 g/kg) malonate; and
b) recovering the malonate from the first aqueous concentrate. The compound of formula I can be referred to as a monosalt of malonic acid and a Group I alkali metal cation (or a ammonium cation), and the compound of formula II can be referred to as a disalt of malonic acid and a Group I alkali metal cations (or ammonium cations). Typically, the Group I alkali metal cation is selected from the group consisting of K⁺, Na⁺, Li⁺, or mixtures thereof, preferably selected from the group consisting of K⁺ and Na⁺ or mixtures thereof, and more preferably the Group I alkali metal cation is K⁺.

As mentioned above, the amount of malonate recovered is measured by the total malonic acid and the malonic acid equivalents contained in the mono- and di-anions of malonic acid recovered, excluding the weight of the cations recovered with the malonate. Malonate is measured by the HPLC method described in the experimental section.

In addition to the methods for recovering malonate from a fermentation broth, the methods described herein also include fermenting a carbohydrate using microbial cells at a suitable pH (as further described below) to produce the fermentation broth containing the malonate to be recovered.

As used herein, the term “fermentation broth” generally refers to a mixture derived from a microbial fermentation process, which contains a medium (liquid; comprising, for example, the produced malonate and other organic acids, salts, metals, residual sugars, and other fermentation by-products) and biomass (solid; comprising, for example, cells and cell debris).

As used herein, the term “fermentation” or “fermenting” generally refers to the feeding of a carbon source (e.g., a sugar, such as glucose) to a microorganism under conditions that enable the microorganism to use the carbon source to produce malonate.

Microorganisms that can be used to produce malonate include any suitable microorganism capable of producing malonic acid from fermentable carbon sources (e.g., glucose, sucrose, and/or other carbohydrates). Representative examples of microorganisms include, but are not limited to, yeasts, such as Saccharomyces cerevisiae, Kluyveromyces lactis, Kluyveromyces marxianus, Yarrowia lipolytica, Pichia kudriavzevii (alternatively referred to as Candida krusei and Issatchenkia orientalis), Schizosaccharomyces pombe, or combinations thereof that are adapted for the production of malonate, for example, genetically engineered versions thereof. In some examples, the host cell can include a microorganism such as a bacterium. Examples of suitable bacteria include Streptococcus, Lactobacillus, Bacillus, Escherichia, Salmonella, Neisseria, Acetobactor, Arthrobacter, Aspergillus, Bifidobacterium, Corynebacterium, Pseudomonas, or a mixture thereof. A genetically modified Pichia kudriavzevii that has been adapted to make significant malonate is particularly preferred for low pH fermenation performance and high malonate tolerance. Preferably, acid tolerant organisms, such as, Kluyveromyces marxianus, Yarrowia lipolytica, Pichia kudriavzevii (alternatively referred to as Candida krusei and Issatchenkia orientalis), Schizosaccharomyces pombe, and filamentous fungi, such as fungi of the genus Aspergillus are utilized. Saccharomyces cerevisiae and Kluyveromyces lactis are preferred in some instances due to the relative ease of conducting genetic modifications to them and the ready availability of genetic tools for modifying them.

The fermentation that provides the malonate recovered using the methods described herein can be conducted under any suitable conditions. For example, a fermenter, can be used to produce a malonate-containing fermentation broth using an organism adapted to make malonate. Such fermentation vessels and associated equipment, such as seed vessels are known in the art. The general fermentation process can utilize any number of commercially available carbohydrate substrates, such carbohydrate substrates comprising high levels of glucose and/or sucrose (for example corn syrups containing at least 40 wt %, at least 50 wt %, at least 60 wt %, at least 70 wt %, at least 80 wt %, at least 85 wt %, at least 90 wt %, or at least 95 wt % glucose by dry weight, and typically less than 99 wt % glucose, for example less than 98 wt %). Fermentation methodology is well-known in the art. For example, the fermentation can be carried out in a batch-wise, fed-batch, continuous or semi-continuous manner.

The fermentation broth during the fermentation typically includes a fermentable carbon source (e.g. the carbohydrate substrates described above), and, typically, a source of nitrogen (such as amino acids, proteins, inorganic nitrogen sources such as urea, ammonia or ammonium salts, and the like), phosphates and sulfates, and additional media components such as vitamins, salts (for example metals), and other materials that can improve cellular growth and/or malonate fermentation production, and water. These components can be fed into a fermenter to conduct the fermentation (e.g. promote growth and production of the malonate product). The microbial culture can be grown under aerobic or micro-aerobic conditions provided by sparging an oxygen containing gas (e.g., air or the like). At the end of the fermentation, all but small quantities of the carbon source have been used by the microorganism, and most of the vitamins and, salts and other materials have been used with the main resulting material at the end of fermenation being malonate, ions (such as metal ions, ammonium ions, sulfates and phosphate), water and cell biomass, organic byproducts (such as organic acid, hydroxyorganic acids and polyols) and other residual materials as further described below.

Typically, at the start or soon after the start of the fermentation, about 3-15 g/L of ammonium sulfate is present; about 1- 6 g/L phosphate anion (e.g. added as potassium dihydrogen phosphate) is present, about 0.25 to 4 g/L magnesium sulfate, trace elements, vitamins and about 60 to 200 g/L of glucose, for example, from about 90 to 180 g/L glucose, from about 130 to 170 g/L glucose. In some instances small quantities of calcium cations may be added during the fermentation. Typically, the amount of calcium cations present in the fermentation broth at the end of the fermentation is less than 1 wt %, preferably less than 0.5 wt %, (for example, less than 0.1 wt %), and more preferably less than 0.05 wt % calcium cations. As discussed in greater detail herein, the pH can be allowed to range freely during the production of malonate, or may be buffered if necessary to prevent the pH from falling below or rising above predetermined levels. Typically, the pH of the fermentation broth at the end of the fermentation can be less than 7.0, less than 6.0, less than 5.0, less than 4.0, less than 3.0, typically from 2.0 to 7.0, from 2.5 to 5.0, from 2.7 to 5.0, from 3.0 to 5.0, from 3.5 to 4.5, from 3.0 to 4.0, or from 3.0 to 3.5. Preferably the pH of the fermentation broth at the end/completion of the fermentation is within 1 pH unit below and 2 pH units above the pH used during the recovery of the malonate, and more preferably within 1 pH unit of the pH used during the recovery of the malonate (for example, if the recovery of the first crop of malonate takes place at a pH of 4.0, then the fermentation pH at the end of fermentation preferably is from 3.0 to 6.0, and more preferably a pH of from 3 to 5). The fermentation can be carried out to allow the pH of the fermentation to drop into a preferred range as malonate is produced. The pH of the fermentation broth can thereafter be maintained within the desired pH range. The desired pH range may vary depending on the extent of the fermentation and the final pH target. For example, the pH may be maintained at a relatively high pH through the addition of basic materials and then allowed to decline as additional malonate is produced until toward the end of the fermentation the desired final pH for the fermentation broth is reached.

The fermentation typically is conducted aerobically or microaerobically, depending on the organism utilized and the requirements of the pathway used to make malonate. If desired, oxygen uptake rate (OUR) can be varied throughout fermentation as a process control. At least partially due to their responsiveness to varying the OUR, crabtree negative organisms, such as crabtree negative yeasts are preferred for use in making malonate. Kluyveromyces marxianus, Yarrowia lipolytica, Pichia kudriavzevii (alternatively referred to as Candida krusei and Issatchenkia orientalis), and Schizosaccharomyces pombe, are examples of crabtree negative yeasts that are preferable to use for the manufacture of malonate.

In one example, the concentration of cells of the organism used to make the malonate typically are present in the fermentation broth at the end of fermentation typically in the range of from 0.5 to 30, from 2 to 30, or from 3 to 20 g dry cells/L of fermentation broth. Typically, these cells are removed prior to recovering the malonate. Preferably, the cells are removed after sufficient water has been removed to create an aqueous solution of typically from 10 to 30 percent by weight malonate, for example from 15 to 30 percent by weight malonate, as further described below. Preferably, after the biomass containing the cells have been removed, additional water is removed to create an aqueous concentrate having at least 400 g malonate/kilogram solution. Alternatively, the first aqueous concentrate can be created by removing the water in a single or multiple steps carried out after the biomass/cells are removed, as described below.

The final yield of malonate produced during the fermentation based on the starting carbon source typically is at least 25%, at least 30%, at least 40%, at least 50%, or greater than 70%. The titer at the completion of the fermentation typically is at least 30 g/L, at least 40 g/L, at least 50 g/L, at least 70 g/L, at least 80 g/L, at least 100 g/L, at least 110 g/L, at least 120 g/L, at least 130 g/L, at least 140 g/L, at least 150 g/L, at least 160 g/L, for example, in a range of from 30 g/L to 300 g/L, from 40 g/L to 300 g/L, from 60 g/L to 250 g/L, from 80 g/L to 220 g/L, or from 100 g/L to 150 g/L, at the end of the fermentation.

The Group I alkali metal cation and/or ammonium cation used in the recovery of the malonate can be added after the fermentation, or at least some or all of the required Group I alkali metal cation and/or ammonium cation can be added during the fermentation. For example, the fermentation broth can include a source of the Group I alkali metal cation, such as a Group I alkali metal carbonate, Group I alkali metal bicarbonate, Group I alkali metal oxide or a Group I alkali metal hydroxide for pH control during the fermentation. The Group I alkali metal carbonate, Group I alkali metal bicarbonate, Group I alkali metal oxide or a Group I alkali metal hydroxide can help maintain the pH of the fermentation broth at the levels described above. A source of ammonium cations such as ammonia, ammonium hydroxide, ammonium bicarbonate, ammonium carbonate and the like can be added as a source of nitrogen for the fermentation and to adjust or maintain the pH during the fermentation.

As used herein, the term “Group I alkali metal cations” generally refers to, for example, cations of lithium (Li), sodium (Na), and potassium (K); that is to Li⁺, Na⁺, and K⁺. Further, as used herein, the term “ammonium cation” includes any cation of the formula R₄N⁺, wherein R is hydrogen or alkyl, wherein “alkyl” refers to groups acyclic or cyclic groups having from 1 to 20 carbon atoms ((C₁-C₂₀)), 10 to 20 carbon atoms ((C₁₀-C₂₀)), 12 to 18 carbon atoms ((C₁₂-C₁₈)), 6 to about 10 carbon atoms ((C₆-C₁₀)), 1 to 10 carbons atoms ((C₁-C₁₀)), 1 to 8 carbon atoms ((C₁-C₈)), 2 to 8 carbon atoms ((C₂-C₈)), 3 to 8 carbon atoms ((C₃-C₈)), 4 to 8 carbon atoms ((C₄-C₈)), 5 to 8 carbon atoms ((C₅-C₈)), 1 to 6 carbon atoms ((C₁-C₆)), 2 to 6 carbon atoms ((C₂-C₆)), 3 to 6 carbon atoms ((C₃-C₆)), or 1 to 3 carbon atoms ((C₁-C₃)). Examples of (C₁-C₂₀)-alkyl groups include acyclic groups such as those with from 1 to 8 carbon atoms such as methyl (i.e., CH₃), ethyl, n-propyl, n-butyl, n-pentyl, n-hexyl, n-heptyl, n-octyl groups. Examples of (C₁-C₂₀)-alkyl groups include cyclic groups such as those with from 1 to 6 carbon atoms such as cyclopropyl, cyclobutyl, cyclopentyl, and cyclohexyl groups.

Suitable sources of the Group I alkali metal cations include, but are not limited to, sodium hydroxide, potassium hydroxide, potassium carbonate, sodium carbonate, and the like. Suitable sources of ammonium cation include, for example, ammonium hydroxide, ammonium carbonate, and the like.

The source of Group I alkali metal cation can also be from compounds that do not necessarily control the pH of the fermentation broth or the aqueous solution used for recovering malonate described herein. For example, the Group I alkali metal cation source can be from compounds such as, sodium chloride, potassium chloride, ammonium chloride, and the like. Regardless of the purpose (e.g., pH control) or properties, the Group I alkali metal cation source can be added to the fermentation broth during or at the end of the fermentation, or to the aqueous solution used for recovering malonate described herein at any time prior to or during the recovery of the malonate.

Removing water from the aqueous solutions (e.g. the fermentation broth and/or the aqueous filtrate) can be performed using any suitable method at any suitable temperature and pressure. In addition, the removing water from the fermentation broth to increase the concentration of the malonate and produce an aqueous concentrate can be implemented two or more times as described in greater detail herein. For example, the water can be removed at less than atmospheric pressure, such as at less than 101.325 kPa. In addition, or alternatively, the water typically can be removed at a temperature (e.g., an aqueous bulk temperature) of 100° C. or less, 90° C. or less, 80° C. or less, preferably 70° C. or less, more preferably 60° C. or less, 50° C. or less; from 40° C. to 100° C., from 50° C. to 90° C., from 50° C. to 80° C., from 50° C. to 70° C. In order to minimize the premature crystallization/precipitation of the malonate, the temperature of the solution containing the malonate is typically maintained at or above 30° C., at least 35° C., at least 40° C., at least 50° C., at least 60° C.; from 30° C. to 100° C., 30° C. to 90° C. or 40° C. to 80° C. if the concentration of malonate is at least 400 g/kg; and preferably, if 500 g/kg or greater malonate concentration, at least 40° C., at least 45° C. The tempearature of the solution containing the malonate preferably is kept lower than 90° C., preferably less than 80° C., and less than 70° C. to minimize decarboxylation of the malonate.

Although a lower limit of about 400 g/kg malonate is described in connection with the removing of water from the aqueous solution obtained from the fermentation broth comprising malonate to increase the concentration of the malonate, the methods described herein also include removing water from the fermentation broth and the aqueous filtrate comprising malonate to enhance the recovery of malonate include increasing the concentration of the malonate to at least 400 g/kg; at least 425 g/kg, at least 450 g/kg; at least 475 g/kg, at least 500 g/kg, at least 525 g/kg, at least 550 g/kg, at least 575 g/kg, at least 600 g/kg; less than 800 g/kg, less than 775 g/kg, such as from 400 g/kg to 775 g/kg, from 450 g/kg to 700 g/kg, and from 500 g/kg to 600 g/kg.

The pH of the aqueous concentrate and/or the aqueous filtrate during the recovery of malonate is typically from 2.5 to 5.0, more preferably from 2.5 to 4.5, from 3.0 to 4.0 (for example from 3.0 to 3.5).

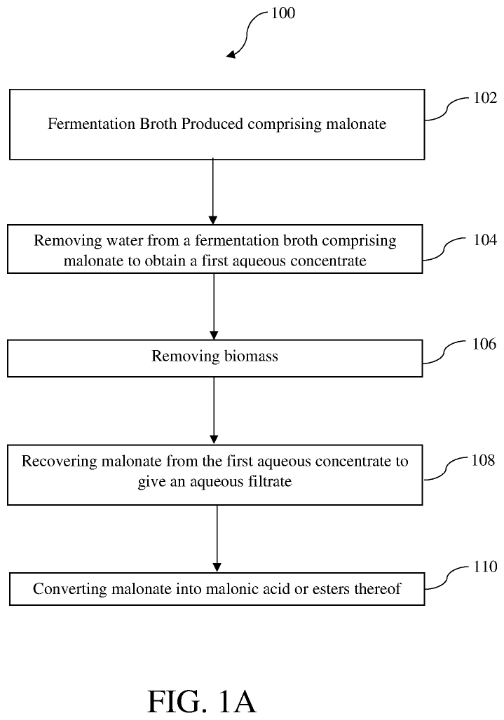

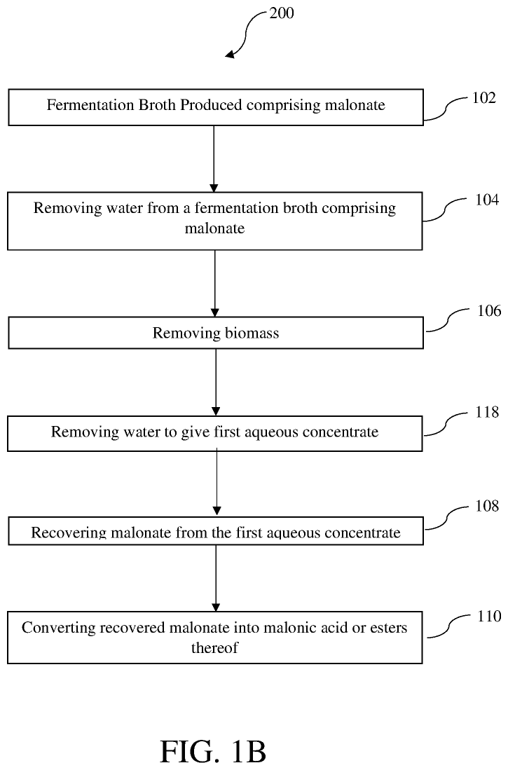

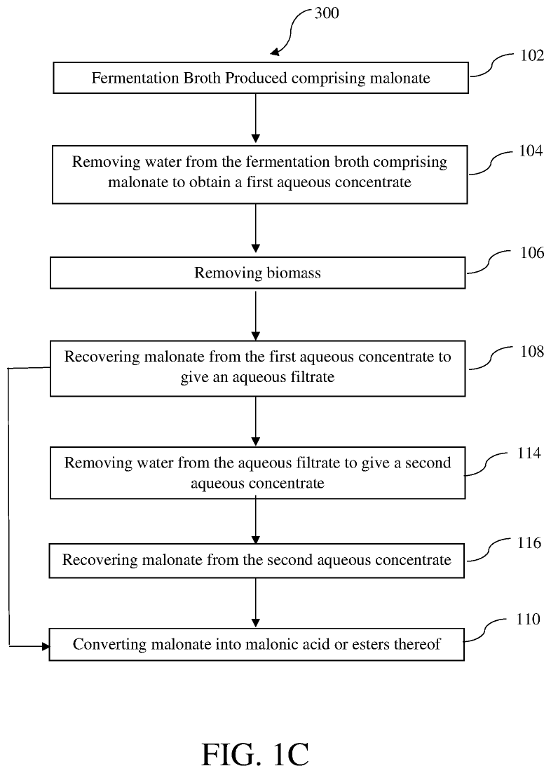

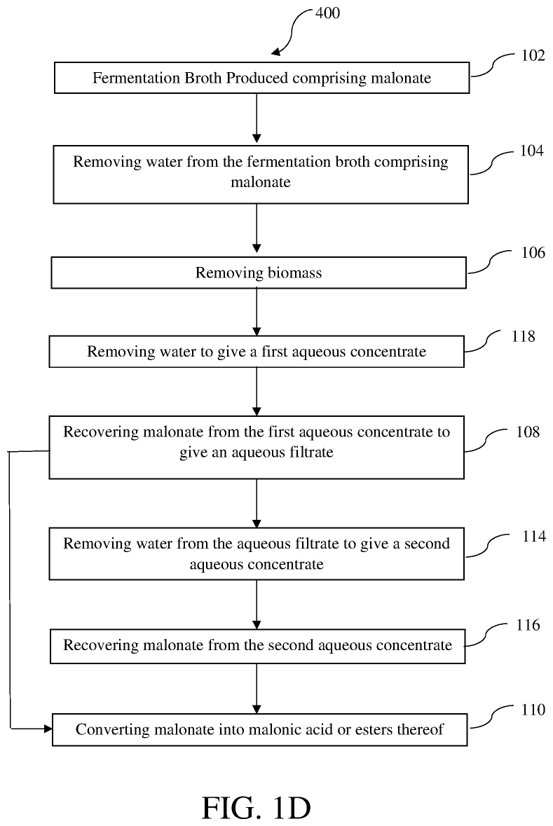

Referring to FIGS. 1A-1D, typically, the biomass (e.g., solid comprising, for example, cells and cell debris) is removed after step 102 or at any point before step 108. Typically, concentration of malonate after the cell removal step is between 30 g/kg and 775 g/kg malonate. Preferably, the cells are removed as shown in FIG. 1B or 1D and the concentration of malonate after removal step typically is from 30 g/kg to 400 g/kg, 50 g/kg to 350 g/kg, 80 g/kg to 350 g/kg. Or the cells are removed as shown in FIG. 1A and the concentration of malonate after removal step typically is 400 g/kg to 800 g/kg, 400 g/kg to 700 g/kg, 500 g/kg to 600 g/kg. For example, the biomass can be removed using one or more filtration steps or one or more centrifugation steps. Centrifugation can be carried out in a decanter centrifuge, such as a horizontal-type decanter centrifuge, a disc stack centrifuge, or hydrocyclones.

As mentioned herein, removing water from the aqueous solution to increase the concentration of the malonate and produce an aqueous concentrate can be implemented two or more times. For example, removing water can comprise removing water from the fermentation broth at step 104 and can further comprise removing water at step 118 to provide a first aqueous concentrate.

Referring to FIGS. 1C and 1D, water is removed in steps 104 and 114 (for FIG. 1C) and in steps 104, 118 and 114 (for FIG. 1D). FIGS. 1C and 1D also show methods for recovering malonate described herein that include recovering the malonate from the first aqueous concentrate and the second aqueous concentrate (e.g., steps 108 and 116) by any suitable method including precipitating or crystallizing The step of recovering the malonate typically includes lowering the temperature of the first aqueous concentrate and/or the second aqueous concentrate to below 40° C., below 35° C., below 30° C., below 25° C., below 20° C., below 15° C., below 10° C., below 5° C., below 0° C.; typically from 0° C. to 30° C., from 4° C. to 25° C. Those of skill in the art will recognize that the temperature at which the malonate can effectively be recovered will depend on the concentration of the malonate in first aqueous concentrate and/or second aqueous concentrate, the pH of the relevant first aqueous concentrate and/or second aqueous concentrate, the number of recovery steps that are utilized, and the desired overall yield of malonate resulting from the recovery steps. For example, the malonate can be recovered at a higher temperature if the relevant aqueous concentrate comprises 700 g/kg of the compound of malonate relative to when the concentration of malonate is 400 g/kg in the aqueous concentrate. However, where a higher yield of malonate is desirable, a lower temperature (typically at or below 30° C. is utilized.

The malonate recovered is typically a mixture of malonic acid, a compound of formula I, and potentially a compound of formula II. For example, for pH's during the recovery of the first crop at a pH between 2.5 and 5.0 (preferably from 2.5 and 4.0, for example from 3.0 to 4.0, from 3.0 to 3.5), and with a second crop recovered without adjusting the pH (i.e. the malonate in the filtrate is concentrated, but no base or acid is added to adjust the pH). Malonate recovered from the first aqueous concentrate typically comprises: at least 50 wt % (for example, at least 55 wt %, at least 60 wt % of the compound of formula 1 (for example from 55 wt % to 75 wt % (from 60 wt % to 75 wt %) of the compound of formula 1; less than 50 wt % (for example, less than 45 wt %, less than 40 wt % (from 1 wt % to 45 wt %, from 2 wt % to 40 wt %, from 20 wt % to 40 wt %)) of malonic acid; and less than 15 wt % (for example, less than 10 wt %, less than 5 wt %, less than 2 wt %, less than 1 wt % (for example from 0.1 wt % to 10 wt %) of the compound of formula II. Malonate recovered from the second aqueous concentrate typically comprises: at least 40 wt % (for example, at least 45 wt %, at least 60 wt % (for example from 40 wt % to 90 wt %, from 60 wt % to 90 wt %) of the compound of formula 1; less than 55 wt % (for example, less than 50 wt %, less than 15 w t% for example, from 1 wt % to 55 wt %, from 1 wt % to 15 wt %) of malonic acid; and less than 25 wt % (for example, less than 20 wt %, less than 17 wt % (for example from 0.05 wt % to 20 wt %, from 1 wt % to 17 wt %)) of the compound of formula II. Typically the total malonate recovered (for example from the sum of the first and the second crop comprises: at least 40 percent (for example at least 50 percent, at least 60 percent) by weight of the compound of formula I; no greater than 25 percent (for example no greater than 20 percent, no greater than 15 percent, no greater than 10 percent, or not greater than 5 percent) by weight of the compound of formula II; and less than 50 percent (less than 45 percent, less than 40 percent, less than 35 percent, less than 30 percent) by weight malonic acid.

Table 1, below provides an example of the typical malonate compositions recovered through the use of the methods described herein:

TABLE 1

| | | | | | | | | | Composite | | | |
|---|---|---|---|---|---|---|---|---|---|---|---|---|
| | 1st Crop | | | | 2nd Crop | | | | (i.e. 1^{st} + 2^{nd} Crops) | | | |
| | | % | % | | | % | % | | | % | % | |
| | % | Mono- | Dipo- | | % | Mono- | Dipo- | | % | Mono- | Dipo- | |
| | Malonic | potassium | tassium | | Malonic | potassium | tassium | Recovery | Malonic | potassium | tassium | Total |
| pH of | Acid | Malonate | Malonate | Recovery | Acid | Malonate | Malonate | Yield of | Acid | Malonate | Malonate | Malonate |
| 1^{st} crop | recov- | recov- | recov- | Yield of | recov- | recov- | recov- | Second | recov- | recov- | recov- | Recov- |
| recovery | ered | ered | ered | 1st Crop¹ | ered | ered | ered | Crop² | ered | ered | ered | ered³ |
| 2.5 | 41.3% | 58.4% | 0.3% | 69.2% | 52.8% | 47.1% | 0.1% | 69.8% | 44.0% | 55.7% | 0.3% | 90.7% |
| 3 | 36.9% | 62.7% | 0.4% | 68.5% | 11.6% | 86.2% | 2.1% | 90.3% | 29.5% | 69.6% | 0.9% | 96.9% |
| 3.5 | 28.3% | 71.1% | 0.6% | 70.9% | 1.5% | 83.3% | 15.2% | 82.7% | 21.5% | 74.2% | 4.3% | 95.0% |
| 4 | 2.8% | 88.0% | 9.2% | 70.9% | 1.6% | 83.8% | 14.6% | 78.8% | 2.5% | 87.0% | 10.5% | 93.8% |

¹Yield based on total recovery of intial malonate
²Yield based on malonate recovery from 1st crop filtrate
³Yield based on malonate recovery in 1st and 2nd crops

The pH of the first aqueous concentrate during the recovery of the first crop typically is as described above. Typically, the pH of the second aqueous concentrate during the recovery of the second crop is not adjusted by the addition of an acid or a base. The pH of the second aqueous concentrate during the recovery of the second crop is typically 0.2 to 1.0 pH unit higher than the pH utilized in the recovery of the first crop, more typically a pH during the second crop that is about 0.2 to about 0.6 higher than the pH utilized during the recovery of the first crop.

Lowering the temperature of the first aqueous concentrate (and the second aqueous concentrate if a second crop is recovered) causes the precipitation of the malonate from the first aqueous concentrate (and the second aqueous concentrate if a second crop is recovered). The malonate can then be removed by, e.g., filtration, to give an aqueous filtrate comprising malonate and a first crop of precipitate of recovered malonate. The aqueous filtrate comprising malonate can, in turn, be concentrated by removing water, as described herein, from the aqueous filtrate to increase the malonate concentration typically to a concentration of from 380 g/kg to 775 g/kg (preferably from 400 g/kg to 700 g/kg, for example from 500 g/kg to 600 g/kg malonate concentration) to form a second aqueous concentrate.

Removing water to form the second aqueous concentrate can be performed using any suitable method at any suitable temperature and pressure. Preferably, the temperatures and pressured described for use in removing water to form the first aqueous concentrate are utilized.

A second crop of precipitate of malonate typically is recovered from the second aqueous concentrate by lowering the temperature of the second aqueous concentrate to similar temperatures as described above for use in recovering the first crop of malonate. The sum of the amounts of malonate recovered in the first crop plus the second crop typically constitute at least 70%, at least 75%, at least 80%, at least 85%, at least 90%, at least 95%; 85% to 99%, 85% to 98% or 90% to 99% of the malonate present in the fermentation broth. If no solvent assisted precipitation is used during recovery of the the first crop or second crop, the recovery of the malonate typically is from 70% to 90% of the malonate present in the fermentation broth. The higher recoveries (e.g. greater than 85%, at least 90%, for example from 90% to 98%, from 93% to 98% of the malonate in the fermentation broth recovered) typically are achieved using solvent assisted precipitation during the first crop and/or second crop recovery.

In theory, a third, fourth or even a fifth crop (or more) of precipitate of malonate can be obtained by continuing to remove water from additional aqueous filtrates that contain malonate by concentration of the malonate, and lowering the temperatures as set forth for the first crop and second crop of malonate precipitate. As mentioned earlier, the precipitation from during any crop may include crystallization or precipitation by other means known to one of ordinary skill in the art.

As mentioned above, the recovery of the compound of the formula I from any of the first aqueous concentrate and/or the second aqueous concentrate (if present) can, in addition to or instead of lowering the temperature, be accomplished by adding an organic solvent. For example, the methods described herein can include adding an organic solvent to the second aqueous concentrate to enhance the recovery of malonate. Preferably, the organic solvent is water-miscible. As mentioned above, preferably the temperature of the aqueous concentrate is reduced to less than 40° C. to enhance the recovery of the malonate. Preferably, a water-miscible organic solvent is added to the second aqueous concentrate to enhance the recovery of malonate from the second aqueous concentrate. In a particular preferred aspect, a water-miscible organic solvent is added to the second aqueous concentrate, but is not added to the first aqueous concentrate. Suitable water-miscible organic solvents include, but are not limited to alcohols (e.g., ethanol), ketones (e.g., acetone), nitriles (e.g., acetonitrile), ethers (e.g., tetrahydrofuran), and combinations thereof. Suitable alcohols include C₁-C₅ alcohols (preferably C₁-C₃), such as methanol, ethanol, propanol, isopropanol, and the like, and combinations thereof. In some examples, ethanol is the water-miscible organic solvent that is added during or prior to step b). In some instances, the water-miscible organic solvent is added prior to recovering the malonate from the first and/or second aqueous concentrate.

Those of skill in the art will recognize that the recovered malonate can be subsequently transformed into a purer form of malonic acid or into a mono- or di-ester of malonic acid of the formula IV:

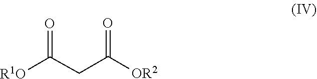

wherein R¹ and R² can be the same or different and can be H, C₁-C₁₀ alkyl (e.g., preferred is C₁-C₆ and C₂-C₆), which is optionally substituted with halo (e.g., fluoro, chloro or bromo), C₄-C₁₀ aryl (e.g., phenyl or naphthyl) or C₁—C₅ alkoxy, provided that R¹ and R² are not both H at the same time.

Examples of the methods contemplated herein are shown in FIGS. 1A-1D. FIG. 1A shows an example method 100, where a fermentation broth is produced in step 102. Step 104 involves removing water from a fermentation broth comprising malonate (e.g., at concentration of at least 30 g/L, at least 40 g/L and less than 200 g/L to increase the concentration of the malonate to at least 400 g/kg) to obtain a first aqueous concentrate. Step 106 involves removing biomass by, e.g., filtration or centrifugation. Step 108 involves recovering malonate as described above from the aqueous concentrate. The malonate can be subsequently converted into malonic acid or esters thereof in step 110. Step 110 is optional.

FIG. 1C is a variation on the method shown in FIG. 1A. FIG. 1C shows an example method 300, where a fermentation broth is produced in step 102. Step 104 involves removing water from a fermentation broth comprising malonate (e.g., at concentration of at least 30 g/L, at least 40 g/L and less than 200 g/L to increase the concentration of the malonate to at least 400 g/kg) to obtain a first aqueous concentrate. Step 106 involves removing biomass by, e.g., filtration or centrifugation. Step 108 involves recovering malonate as described above from the first aqueous concentrate by, e.g., precipitation and filtration to give an aqueous filtrate. In step 114 water is removed from the aqueous filtrate to give a second aqueous concentrate. Step 116 involves recovering malonate as described above from the second aqueous concentrate. The malonate recovered during the first crop and the second crop can be subsequently converted into malonic acid or esters thereof in step 110. Step 110 is optional.

FIG. 1B shows an example method 200, where a fermentation broth is produced in step 102. Step 104 involves removing water from a fermentation broth. Step 106 involves removing biomass by, e.g., filtration or centrifugation. Step 118 involves removing water from the material obtained after step 106 to give a first aqueous concentrate. Step 108 involves recovering malonate as described above from the first aqueous concentrate. The malonate can be subsequently converted into malonic acid or esters thereof in step 110. Step 110 is optional.

FIG. 1D shows an example method 400, where a fermentation broth is produced in step 102. Step 104 involves removing water from a fermentation broth. Step 106 involves removing biomass by, e.g., filtration or centrifugation. Step 118 involves removing water from the material obtained after step 106 to give a first aqueous concentrate. Step 108 involves recovering malonate as described above from the first aqueous concentrate by, e.g., precipitation and filtration to give an aqueous filtrate. In step 114 water is removed from the aqueous filtrate to give a second aqueous concentrate. Step 116 involves recovering malonate from the second aqueous concentrate. The malonate recovered from the first and the second crops can be subsequently converted into malonic acid or esters thereof in step 110. Step 110 is optional.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “0.1% to 5%” or “0.1% to 5%” should be interpreted to include not just 0.1% to 5%, but also the individual values (e.g., 1%, 2%, 3%, and 4%) and the sub-ranges (e.g., 0.1% to 0.5%, 1.1% to 2.2%, 3.3% to 4.4%) within the indicated range.

In this document, the terms “a,” “an,” or “the” are used to include one or more than one unless the context clearly dictates otherwise. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately.

The term “about” as used herein can allow for a degree of variability in a value or range, for example, within 10%, within 5%, or within 1% relative to a stated value or of a stated limit of a range, and includes the exact stated value or range.

The term “substantially” as used herein refers to a majority of, or mostly, as in at least about 96%, 97%, 98%, 99%, 99.5%, 99.9%, 99.99%, or at least about 99.999% or more, or 100%.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

### EXAMPLES

The present invention can be better understood by reference to the following examples which are offered by way of illustration. The present invention is not limited to the examples given herein.

### General Methods

In the examples described herein, the samples were analyzed via high performance liquid chromatography (HPLC) using a guard column (Bio-Rad Cation H+ cartridge (p/n 125-0129) and holder (p/n 125-0131)); and an analytical column (2x Bio-Rad Aminex HPX-87H 300 mm×7.8 mm columns (125-0140)) in series. The injection volume for all runs was 10 μL. The mobile phase was isocratic (30 mM H₂SO₄). The column temperature was held at 55° C. during each chromatography run. The flow rate was maintained at 0.6 mL/min, with each run duration being 45 minutes. Two detectors were used, namely, a refractive index detector (RID) (cell temperature held at 40° C.) and an ultraviolet detector (UVD) with the detector wavelength set at 210 nm. “Malonic acid equivalents” refers to the total malonates (i.e. free malonic acid, mono-salts of malonic acid, and di-salts of malonic acid, expressed as malonic acid). The malonic acid equivalents and malonate content is determined using the HPLC methods described above.

### Example 1: Precipitation of Malonate at pH 3 from a Malonate Containing Solution

To a one-liter reaction flask equipped with a multi-neck lid, impeller and pH probe, add 200 g malonic acid and 125 g of water. Agitation is started by ramping up to 250 RPM over 3 seconds. Potassium hydroxide pellets are added over a 20-minute period to bring the pH to 3. The mass of potassium hydroxide needed to do this is recorded (the mass is between 70 and 75 g). Once a pH of 3 is reached, the temperature is increased to 70° C. over a 5-minute period. The temperature is then decreased to 8° C. or 16° C. at a rate of 1° C./minute. Once a temperature of 8° C. or 16° C. is reached, the reaction is held at that temperature for 120 minutes. Crystal/precipitate formation occurs between 20 and 25° C. After 120 minutes of holding at the designated temperature, the reaction mixture is filtered. The mass of the crystals/precipitate after filtration and the filtrate are both recorded. The crystals/precipitate are dried to constant weight in an oven at 40° C. and reweighed. Both the crystals/precipitate and the filtrate are analyzed by HPLC to determine the amount of malonate present. Percent recovery is determined for the solid and mass balance is calculated. Recovery of 67% malonate in crystals. The percentage of malonic acid, mono-potassium malonate, and di-potasium malonate are similar to those shown in Table 1 for a pH of 3.0.

### Example 2: Precipitation of Malonate at pH 4 from a Malonate Containing Solution

To a one-liter reaction flask equipped with a multi-neck lid, impeller and pH probe, add 200 g malonic acid and 90.95 g of water. Agitation is started by ramping up to 250 RPM over 3 seconds. Potassium hydroxide pellets are added over a 20-minute period to bring the pH to 4. The mass of potassium hydroxide needed to do this is recorded (the mass is between 105 and 110 g). Once a pH of 4 is reached, the temperature is increased to 70° C. over a 5-minute period. The temperature is then decreased to 8° C. at a rate of 1° C./minute. Once a temperature of 8° C. is reached, the reaction is held at 8° C. for 120 minutes. Crystal/precipitate formation occurs between 20 and 25° C. After 120 minutes of holding at 8° C., the reaction mixture is filtered. The mass of the crystals/precipitate after filtration and the filtrate are both recorded. The crystals/precipitate are dried to constant weight in an oven at 40° C. and reweighed. Both the crystals/precipitate and the filtrate are analyzed by HPLC to determine the amount of malonate present. Percent recovery is determined for the solid and mass balance is calculated. Recovery of 72% malonate in crystals/precipitate. The percentage of malonic acid, mono-potassium malonate, and di-potasium malonate are similar to those shown in Table 1 for a pH of 4.0.

### Example 3: Precipitation of Malonate at pH 3 from a Malonate Containing Solution

To a one-liter reaction flask equipped with a multi-neck lid, impeller and pH probe, were added the following components likely to be present in an actual fermentation broth used to produce malonic acid. A “mock broth” containing the following components: 400 g malonic acid, 4 g of succinic acid, 4 g of 3-hydroxypropionic acid, 2 g sodium pyruvate, 8 g glucose, 0.8 g maltose, 4 g arabitol, 0.6 g ammonium phosphate, and 230 g of water is produced. Agitation is started by ramping up to 250 RPM over 3 seconds. Potassium hydroxide pellets are added over a 20-minute period to bring the pH to 3. The mass of potassium hydroxide needed to do this is recorded (the mass is between 140 and 150 g). Once a pH of 3 is reached, the temperature is increased to 70° C. over a 5-minute period. The temperature is then decreased to 8° C. or 6° C. at a rate of 1° C./minute. Once a temperature of 8° C. or 6° C. is reached, the reaction is held at that temperature for 120 minutes. Crystals/precipitate formation occurs between 20 and 25° C. After 120 minutes of holding at the set temperature, the reaction mixture is filtered. The mass of the crystals/precipitate after filtration and the filtrate are both recorded. The crystals/precipitate are dried to constant weight in an oven at 40° C. and reweighed. Both the crystals/precipitate and the filtrate are analyzed by HPLC to determine the amount of malonate and other components that are present. % recovery is determined for the solid and mass balance is calculated. Recovery of 67-71% malonate in crystals/precipitate. The percentage of malonic acid, mono-potassium malonate, and di-potasium malonate are similar to those shown in Table 1 for a pH of 3.0.

### Example 4: Solvent Precipitation of Malonate from Concentrated Filtrate

The filtrate generated by precipitation of malonate from the mock broth of Example 3 is analyzed on a halogen moisture balance to determine % dry solids content. The % dry solids content value is used to determine the amount of water to remove to bring the filtrate to a 60% or 70% dry solids content. The filtrate is concentrated to the desired % dry solids content value via rotary evaporation at 60° C. and 70 torr. The concentrated filtrate and the condensate are analyzed via HPLC.

A weighed portion of the evaporated filtrate is placed in a reaction flask equipped with a multi-neck lid, impeller and pH probe. Ethanol is added at a 2:1, 4:1, or 6:1 w:w ratio based on the amount of malonic acid in the filtrate. Agitation is started by ramping up to 250 RPM over 3 seconds. The hold temperature (6° C., 8° C., or 25° C.) is set to be reached as soon as possible, and the mixture is held at that temperature for 30 minutes. After 30 minutes, the crystals/precipitate are filtered on a Pannevis apparatus. Both the mass of the wet crystals/precipitate and the filtrate are obtained. The crystals/precipitate are dried to constant weight at 40° C. in an oven and reweighed. The solid and the filtrate are both analyzed by HPLC to determine the amount of malonate present. Percent recovery is determined for the solid and mass balance is calculated. The total percent recovery of malonate is determined for the two steps. Malonate recovery of 90-97% for the two steps (i.e. two crops). The percentage of malonic acid, mono-potassium malonate, and di-potasium malonate recovered in this example 4 are similar to the malonate recovered during a second crop shown in Table 1, when the pH used to recovery the first crop was pH of 3.0.

### Example 5: Precipitation of Malonate at pH 4 from a Mock Broth Solution

To a one liter reaction flask equipped with a multi-neck lid, impeller and pH probe, the following are added to prepare a mock broth: malonic acid (200 g), succinic acid (2 g), 3-hydroxypropionic acid (2 g), sodium pyruvate (1 g), glucose (4 g), maltose (0.4 g), arabitol (2 g), ammonium phosphate (0.3 g), and water (79.4 g). Agitation is started by ramping up to 250 RPM over 3 seconds. Potassium hydroxide pellets are added over a 20-minute period to bring the pH to 4. The mass of potassium hydroxide needed to do this is recorded (the mass is between 90 and 100 g). Once a pH of 4 is reached, the temperature is increased to 70° C. over a 5-minute period. The temperature is then decreased to 8° C. at a rate of 1° C./minute. Once a temperature of 8° C. is reached, the reaction is held at that temperature for 120 minutes. Crystal/precipitate formation occurs between 20 and 25° C. After 120 minutes of holding at the set temperature, the reaction mixture is filtered. The mass of the crystals/precipitate after filtration and the filtrate are both recorded. The crystals/precipitate are dried to constant weight at 40° C. in an oven and reweighed. Both the crystals/precipitate and the filtrate are analyzed by HPLC to determine the amount of malonate and other components that are present. Percent recovery is determined for the solid and mass balance is calculated. Recovery of 71% malonate in crystals/precipitate. The percentage of malonic acid, mono-potassium malonate, and di-potasium malonate are similar to those shown in Table 1 for a pH of 4.0.

### Example 6: Solvent Precipitation of Malonate at pH 3 from a Malonate Containing Solution

To a one-liter reaction flask equipped with a multi-neck lid, impeller and pH probe, malonic acid (200 g) and water (125.1 g) are combined. Agitation is started by ramping up to 250 RPM over 3 seconds. Potassium hydroxide pellets are added over a 20-minute period to bring the pH to 3. The mass of potassium hydroxide needed to do this is recorded (the mass is between 70 and 80 g). While potassium hydroxide is being added, the temperature is increased to 50° C. Once potassium hydroxide addition is complete and a temperature of 50° C. is reached, ethanol (in a 2:1, 1:1, or 0.5:1 w:w ethanol to malonic acid) is added. After ethanol addition is completed, the temperature is decreased to 25° C. at a rate of 1° C./min Once a temperature of 25° C. is reached, that temperature is maintained for 2 hours. The temperature at which precipitation occurs is dependent on the ratio of ethanol to malonic acid. Once the temperature has been held for 2 hours, the crystals/precipitate are filtered on a Pannevis apparatus. Both the mass of the wet crystals/precipitate and the filtrate are obtained. If there are crystals visible in the filtrate, water is added to the filtrate until no solids remain, and the mass of water used is recorded. Dilution of the filtrate was performed for analytical purposes. The crystals/precipitate are dried to constant weight at 40° C. in an oven and reweighed. The solid and the filtrate are both analyzed by HPLC to determine the amount of malonate present. Recovery of 68-73% malonate in crystals/precipitate. The percentage of malonic acid, mono-potassium malonate, and di-potasium malonate are similar to those shown in Table 1 for a pH of 3.0.

### Example 7: Solvent Precipitation of Malonate at pH 4 from a Malonate Containing Solution

To a one-liter reaction flask equipped with a multi-neck lid, impeller and pH probe, malonic acid (200 g) and water (90.95 g) are combined. Agitation is started by ramping up to 250 RPM over 3 seconds. Potassium hydroxide pellets are added over a 20-minute period to bring the pH to 4. The mass of potassium hydroxide needed to do this is recorded (the mass is between 100 and 110 g). While potassium hydroxide is being added, the temperature is increased to 50° C. Once potassium hydroxide addition is complete and a temperature of 50° C. is reached, ethanol (in a 1:1, 0.5:1, or 0.25:1 w:w ethanol to malonic acid) is added. After ethanol addition is completed, the temperature is decreased to 25° C. at a rate of 1° C./min. Once a temperature of 25° C. is reached, that temperature is maintained for 2 hours. The temperature at which precipitation occurs is dependent on the ratio of ethanol to malonic acid. Once the temperature has been held for 2 hours, the crystals are filtered on a Pannevis apparatus. Both the mass of the wet crystals/precipitate and the filtrate are obtained. If there are crystals/precipitate visible in the filtrate, water is added to the filtrate until no solids remain, and the mass of water used is recorded. Dilution of the filtrate was performed for analytical purposes. The crystals/precipitate are dried to constant weight at 40° C. in an oven and reweighed. The solid and the filtrate are both analyzed by HPLC to determine the amount of malonate present. Recovery of 52-77% malonate in crystals/precipitate, yield varied based on amount of ethanol added. The percentage of malonic acid, mono-potassium malonate, and di-potasium malonate are similar to those shown in Table 1 for a pH of 4.0.

### Example 8: Solvent Precipitation of Malonate at pH 3 from a Mock Broth Solution using a 2:1 Weight:Weight Ethanol:Malonate Weight Ratio

To a one-liter reaction flask equipped with a multi-neck lid, impeller and pH probe, the following are added to prepare a mock broth: malonic acid (200 g), succinic acid (2 g), 3-hydroxypropionic acid (2 g), sodium pyruvate (1 g), glucose (4 g), maltose (0.4 g), arabitol (2 g), ammonium phosphate (0.3 g), and water (79.4 g). Agitation is started by ramping up to 250 RPM over 3 seconds. Potassium hydroxide pellets are added over a 20-minute period to bring the pH to 3. The mass of potassium hydroxide needed to do this is recorded (the mass is between 70 and 80 g). While potassium hydroxide is being added, the temperature is increased to 50° C. Once potassium hydroxide addition is complete and a temperature of 50° C. is reached, ethanol (400 g) is added quickly. After ethanol addition is completed, the temperature is decreased to 25° C. at a rate of 1° C./min. Once a temperature of 25° C. is reached, that temperature is maintained for 2 hours. Crystals/precipitate began to form during addition of ethanol. Once the temperature has been held for 2 hours, the crystals/precipitate are filtered on a Pannevis apparatus. Both the mass of the wet crystals/precipitate and the filtrate are obtained. If there are crystals/precipitate visible in the filtrate, water is added to the filtrate until no solids remain, and the mass of water used is recorded. Dilution of the filtrate was performed for analytical purposes. The crystals/precipitate are dried to constant weight at 40° C. in an oven and reweighed. Recovery of 72% malonate in crystals/precipitate. The percentage of malonic acid, mono-potassium malonate, and di-potasium malonate are similar to those shown in Table 1 for a pH of 3.0.

### Example 9: Solvent Precipitation of Malonate at pH 3 from a Mock Broth Solution using a 4:1 Weight:Weight Ethanol:Malonate Weight Ratio

To a one-liter reaction flask equipped with a multi-neck lid, impeller and pH probe, the following are added to prepare a mock broth: malonic acid (125 g), succinic acid (1.25 g), 3-hydroxypropionic acid (1.25 g), sodium pyruvate (0.625 g), glucose (2.5 g), maltose (0.25 g), arabitol (1.25 g), ammonium phosphate (0.1875 g), and water (71.9 g). Agitation is started by ramping up to 250 RPM over 3 seconds. Potassium hydroxide is added over a 20-minute period to bring the pH to 3. The mass of potassium hydroxide needed to do this is recorded (the mass is between 40 and 50 g). While potassium hydroxide is being added, the temperature is increased to 50° C. Once potassium hydroxide addition is complete and a temperature of 50° C. is reached, ethanol (400 g) is added quickly. After ethanol addition is completed, the temperature is decreased to 25° C., 16° C., or 8° C. at a rate of 1° C./min. Once a temperature of 25° C., 16° C., or 8° C. is reached, that temperature is maintained for 2 hours. Crystals/precipitate began to form during addition of ethanol. Once the temperature has been held for 2 hours, the crystals/precipitate are filtered on a Pannevis apparatus. Both the mass of the wet crystals/precipitate and the filtrate are obtained. If there are crystals/precipitate visible in the filtrate, water is added to the filtrate until no solids remain, and the mass of water used is recorded. Dilution of the filtrate was performed for analytical purposes. The crystals are dried to constant weight at 40° C. in an oven and reweighed. Recovery of 73-80% malonate in crystals/precipitate. The percentage of malonic acid, mono-potassium malonate, and di-potasium malonate are similar to those shown in Table 1 for a pH of 3.0.

1. A method for recovering a malonate comprising the compound of formula I: 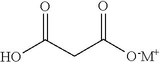
wherein M is a Group I alkali metal,
optionally comprising the compound of formula II: 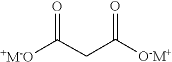
wherein M is a Group I alkali metal, and
optionally comprising malonic acid, the method comprising the steps of:
a) removing water from a fermentation broth comprising malonate, to obtain a first aqueous concentrate having a malonate concentration of at least about 400 g/kg; and
b) recovering the malonate from the first aqueous concentrate, wherein the malonate comprises: at least 40 percent by weight of the compound of formula I; no greater than 25 percent by weight of the compound of formula II; and less than 50 percent by weight malonic acid.

2. The method of claim 1, wherein the recovering comprises lowering the temperature during step b) to below 40° C.

3. The method of claim 1, wherein the pH of the first aqueous concentrate before or at step b) is from 2.5 to 5.0.

4. The method of claim 1, further comprising a step of:
c) removing biomass before step b).

5. The method of claim 1, wherein step b) further comprises:
adding a water-miscible organic solvent to the first aqueous concentrate.

6. The method of claim 4, wherein step c) comprises using a centrifuge or filter to remove the biomass.

7. The method of claim 4, wherein the concentration of malonate after step c) is from about 40 g/kg to about 400 g/kg.

8. The method of claim 4, wherein the concentration of malonate at the beginning of step b) is from about 400 g/kg to about 700 g/kg.

9. The method of claim 4, wherein the step a) comprises a1) removing water from the fermentation broth prior to step c); and a2) removing water from the fermentation broth after step c).

10. The method of claim 1, wherein step a) comprises removing water from the fermentation broth at an aqueous solution bulk temperature of less than 100° C.

11. The method of claim 1, wherein step a) comprises removing water at less than atmospheric pressure.

12. The method of claim 2, wherein the temperature prior to step b) is at least 30° C. and the concentration of malonate is at least 400 g/kg.

13. The method of claim 1, wherein the temperature during recovery step b) is from about 0° C. to about 30° C.

14. The method of claim 1, wherein the pH of the fermentation broth at the end of fermentation is within 2 pH units of the pH of the first aqueous concentrate produced in step a).

15. The method of claim 1, wherein step b) comprises precipitating or crystallizing the malonate from the first aqueous concentrate.

16. The method of claim 15, wherein the malonate is recovered and an aqueous filtrate is produced comprising malonate.

17. The method of claim 16, further comprising removing water from the aqueous filtrate to increase the malonate concentration to form a second aqueous concentrate having a malonate concentration of from about 380 g/kg to about 750 g/kg.

18. The method of claim 17, further comprising:
d) recovering malonate from the second aqueous concentrate, the recovered malonate comprising at least 40 wt % of the compound of formula 1; less than 55 wt % of malonic acid; and less than 25 wt % of the compound of formula II.

19. (canceled)

20. The method of claim 18, wherein step d) comprises adding a water-miscible organic solvent.

21. (canceled)

22. The method of claim 1, wherein the Group I alkali metal cation comprises Na⁺, K⁺, Li⁺, or combinations thereof.

23. The method of claim 1, wherein the Group I alkali metal cation comprises K⁺.

24-42. (canceled)

43. The method of any of claim 18, wherein malonate recovered from the first aqueous concentrate and the second aqueous concentrate is at least 80%, of the malonate in the fermentation broth recovered.

44-47. (canceled)
